(19) United States
(12) Patent Application Publication
(10) Pub. No.: US 2018/0243327 A1
(43) Pub. Date: Aug. 30, 2018

(54) USE OF SECOISOLARICIRESINOL DIGLUCOSIDES (SDGs) AND RELATED COMPOUNDS FOR PROTECTION AGAINST RADIATION DAMAGE

(71) Applicant: THE TRUSTEES OF THE UNIVERSITY OF PENNSYLVANIA, Philadelphia, PA (US)

(72) Inventor: Melpo CHRISTOFIDOU-SOLOMIDOU, Eagleville, PA (US)

(73) Assignee: THE TRUSTEES OF THE UNIVERSITY OF PENNSYLVANIA, Philadelphia, PA (US)

(21) Appl. No.: 15/757,273

(22) PCT Filed: Aug. 31, 2016
(86) PCT No.: PCT/US16/49780

§ 371 Date: Mar. 2, 2018

Related U.S. Application Data
(60) Provisional application No. 62/212,898, filed on Sep. 1, 2015.

(51) Int. Cl. A61K 31/7032; A61K 9/00; A61P 39/00; A61K 36/55; A61K 31/05; A61K 31/365; A61P 11/00; A23L 33/105

(52) U.S. Cl. CPC A61K 31/7032; A61K 9/0053; A61P 39/00; A61K 36/55; A23V 2002/00; A61K 31/365; A61P 11/00; A23L 33/105; A61K 31/05

(57) ABSTRACT

The invention relates to the use of secoisolariciresinol diglucoside (SDG), other active components in flaxseed, and related compounds for treating proton radiation associated lung injury and protecting normal lung tissue against proton radiation exposure. The invention also relates to the use of SDG, other active components in flaxseed, and related compounds in down-regulating senescence markers, and thereby protecting from senescence associated or radiation induced aging phenotypes.

### GOVERNMENT INTEREST

This invention was made with government support under Grant Numbers NIH-R01 CA133470, NIH-1R21NS087406-01 and NIH-R03 CA180548 awarded by the National Institute of Health of the United States Department of Health and Human Services. The government has certain rights in the invention.

### FIELD OF THE INVENTION

The invention relates to the use of secoisolariciresinol diglucoside (SDG), obtained from natural sources, such as flaxseed, or generated synthetically (synthetic SDG is also referred to herein as LGM 2605), other active components in flaxseed, secoisolariciresinol (SECO), enterodiol (ED), and enterolactone (EL), as well as stereoisomers of the foregoing, metabolites of the foregoing, degradants of the foregoing, and analogs of the foregoing, for treating proton radiation associated lung injury and protecting normal lung tissue against proton radiation exposure. The invention also relates to the use of SDG, other active components in flaxseed, and related compounds in down-regulating senescence markers, and thereby protecting from senescence associated aging phenotypes.

### BACKGROUND OF THE INVENTION

Ionizing radiation produces a wide range of deleterious effects in living organisms. Humans are exposed to radiation as an occupational hazard, during diagnostic and therapeutic radiographic procedures, when using electronic devices, from background radiation of nuclear accidents, during air and space travel, as well as from prolonged exposure to the sun (e.g., sun bathers or outdoor workers). Exposure to natural radiation can occur in many forms: natural resources such as air, water, and soil may become contaminated when they come in contact with naturally-occurring, radiation-emitting substances (radionuclides); radon is one such common source of natural radiation. Current global developments have additionally established terrorism as a dangerous means by which potentially large numbers of people can be exposed to lethal amounts of radiation. It is, therefore, of high importance to identify agents that can be administered before and during exposure to radiation (i.e., radioprotective agents), and as treatment after radioactive exposure (i.e., radiation mitigators).

Lung cancer remains the leading cause of death from cancer in the US and worldwide. Introduction of proton radiation therapy, led to the reduction of the dose received by the tumor-surrounding normal tissue while allowing more focused doses to the tumor target. Nevertheless, a substantial risk of late side effects in long term survivors, such as significant normal tissue damage, still remains.

Clinically significant radiation lung injury, such as pneumonia-like inflammation and late stage fibrosis, occurs in up to 30% of patients irradiated for lung cancer and about 10-15% of other thoracic oncology patients. The need, however, to protect “normal” lung parenchyma from unacceptable radiation injury compromises the ability to deliver tumoricidal radiotherapy doses and contributes to the high local recurrence rates experienced by lung cancer patients following definitive radiotherapy. The cytotoxic effects of ionizing radiation in normal lung parenchyma are mediated by the generation of reactive oxygen species (ROS) and propagated by ROS-driven oxidative stress thus identifying a central role of tissue antioxidant defense. A safe radioprotecting agent that would ameliorate radiation toxicity while not protecting tumor, or even preferably radiosensitizing tumor cells is desperately needed.

In addition, there exists a need to protect radiation associated diseases such as aging and other conditions.

### SUMMARY OF THE INVENTION

In one aspect, the invention provides a method for treating or preventing radiation damage in a subject who has been or will be exposed to radiation (e.g., proton radiation), the method comprising: administering to said subject an effective amount of secoisolaricirecinol diglucoside (SDG), an SDG analog, an SDG stereoisomer, or a combination thereof.

In another aspect, the invention provides a method for treating or preventing proton radiation associated lung injury in a subject in need thereof, the method comprising: administering to said subject an effective amount of secoisolaricirecinol diglucoside (SDG), an SDG analog, an SDG stereoisomer, or a combination thereof, thereby treating or preventing said proton radiation associated lung injury in said subject.

In another aspect, the invention provides a method for protecting normal lung tissue against proton radiation exposure in a subject in need thereof, the method comprising: administering to said subject an effective amount of secoisolaricirecinol diglucoside (SDG), an SDG analog, an SDG stereoisomer, or a combination thereof, thereby protecting normal lung tissue against proton radiation exposure in said subject.

In another aspect, the invention provides a method for protecting a biomolecule, a cell, or a tissue from damage by proton radiation in a subject in need thereof, the method comprising: administering to said subject an effective amount of secoisolaricirecinol diglucoside (SDG), an SDG analog, an SDG stereoisomer, or a combination thereof.

In another aspect, the invention provides a method for treating or preventing radiation damage in a subject who has been or will be exposed to radiation, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), analogs thereof, stereoisomers thereof, or a combination thereof.

In another aspect, the invention provides a method for treating or preventing proton radiation associated lung injury in a subject in need thereof, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), analogs thereof, stereoisomers thereof, or a combination thereof.

In another aspect, the invention provides a method for protecting normal lung tissue against proton radiation exposure in a subject in need thereof, the method comprising: administering to said subject an effective amount of at least one bioactive ingredient, wherein said bioactive ingredient comprises secoisolariciresinol diglucoside (SDG), other active components in flaxseed, secoisolariciresinol (SECO), enterodiol (ED), and enterolactone (EL), as well as stereoisomers of the foregoing, metabolites of the foregoing, degradants of the foregoing, analogs of the foregoing, or a combination of the foregoing.

In another aspect, the invention provides a method for protecting a biomolecule, a cell, or a tissue from damage by proton radiation in a subject in need thereof, the method comprising: administering to said subject an effective amount of at least one bioactive ingredient, wherein said bioactive ingredient comprises secoisolariciresinol diglucoside (SDG), other active components in flaxseed, secoisolariciresinol (SECO), enterodiol (ED), and enterolactone (EL), as well as stereoisomers of the foregoing, metabolites of the foregoing, degradants of the foregoing, analogs of the foregoing, or a combination of the foregoing.

In another aspect, the invention provides a method for treating or preventing a senescent phenotype in a subject in need thereof, the method comprising: administering to said subject an effective amount of secoisolaricirecinol diglucoside (SDG), an SDG analog, an SDG stereoisomer, or a combination thereof, thereby treating said senescent phenotype in said subject.

In another aspect, the invention provides a method for treating or preventing a senescence associated aging disease or condition in a subject in need thereof, the method comprising: administering to said subject an effective amount of secoisolaricirecinol diglucoside (SDG), an SDG analog, an SDG stereoisomer, or a combination thereof, thereby treating said senescence associated aging disease or condition in said subject.

In another aspect, the invention provides a method for treating or preventing a senescent phenotype in a subject in need thereof, the method comprising: administering to said subject an effective amount of at least one bioactive ingredient, wherein said bioactive ingredient comprises secoisolariciresinol diglucoside (SDG), other active components in flaxseed, secoisolariciresinol (SECO), enterodiol (ED), and enterolactone (EL), as well as stereoisomers of the foregoing, metabolites of the foregoing, degradants of the foregoing, analogs of the foregoing, or a combination of the foregoing.

In another aspect, the invention provides a method for treating or preventing a senescence associated aging disease or condition in a subject in need thereof, the method comprising: administering to said subject an effective amount of at least one bioactive ingredient, wherein said bioactive ingredient comprises secoisolariciresinol diglucoside (SDG), other active components in flaxseed, secoisolariciresinol (SECO), enterodiol (ED), and enterolactone (EL), as well as stereoisomers of the foregoing, metabolites of the foregoing, degradants of the foregoing, analogs of the foregoing, or a combination of the foregoing.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1. Bioactive polyphenolic plant lignans in flaxseed and radiation Damage. Secoisolariciresinol diglucoside (SDG) is the major lignan phenolic in flaxseed. Due to complex SDG extraction, purification and enrichment methods from natural resources, SDG was chemically synthesized. Synthetic SDG (LGM2605) has strong antioxidant and free radical, scavenging characteristics. (B) The structure of SDG.

FIG. 2: Novel Ex vivo model of human lung: human precision-cut lung slices for radiation exposure. Human Precision-cut slice preparation: Slices (huPCLS) were obtained by inflating the donor human lung with low melting temperature agarose, sectioning, coring, and slicing, as shown above. The 350 μm thick slices were washed three times with culture medium to rid airways of agarose and are viable at 37 oC, up to a week.

FIG. 3: Evaluation of inflammatory markers, in huPCLS, at 30 min and 24 hours post 4 Gy proton radiation with or without SDG pre-treatment for 4 hours. Transcript levels of (A) IL1B, (B) IL6 and (C) TNF-alpha are normalized to 18S rRNA. *p<0.05. FIG. 3 shows that LGM2605 Prevents the Induction of Proinflammatory Cytokine gene levels by Proton Radiation, in huPCLS.

FIG. 4: qPCR analysis of (A) HMOX-1 and (B) NQO1 in huPCLS after 30 min and 24 hours post 4 Gy proton radiation with or without SDG pretreatment for 4 hours. Data are represented as average fold change from non-irradiated control±SEM. Transcript levels of tested genes are normalized to 18S rRNA. *p<0.05. FIG. 4 shows that LGM2605 Boosts Antioxidant Gene Levels by Proton Radiation in huPCLS

FIG. 5: LGM2605 reduces senescent-like phenotype of huPCLS. FIG. 5 shows that prevention of senescent phenotype after SDG treatment. A) SA-β-Gal staining of huPCLS at 24 hour post proton radiation. B) Quantification of the β-gal positive stained cells. Percentage change of the β-gal positive cells of the irradiated huPCLS compared to the nonirradiated huPCLS.

FIG. 6: LGM2605 decreases Proton Radiation-induced Senescence Markers, both at the Gene and Protein Level, in huPCLS. FIG. 6A shows schematic representation of SDG (LGM2605) blockade of senescence markers induced by proton radiation of normal lung tissue/cell. FIG. 6B shows the reduction of senescence markers after SDG treatment-qPCR analysis in huPCLS after 30 min and 24 hours post 4 Gy proton radiation with or without SDG pretreatment for 4 hours. Transcript levels of tested genes are normalized to 18S rRNA.*p<0.05. FIG. 6C shows Western Blot analysis of whole tissue lysates from huPCLS treated with proton. FIG. 6D shows summary of action.

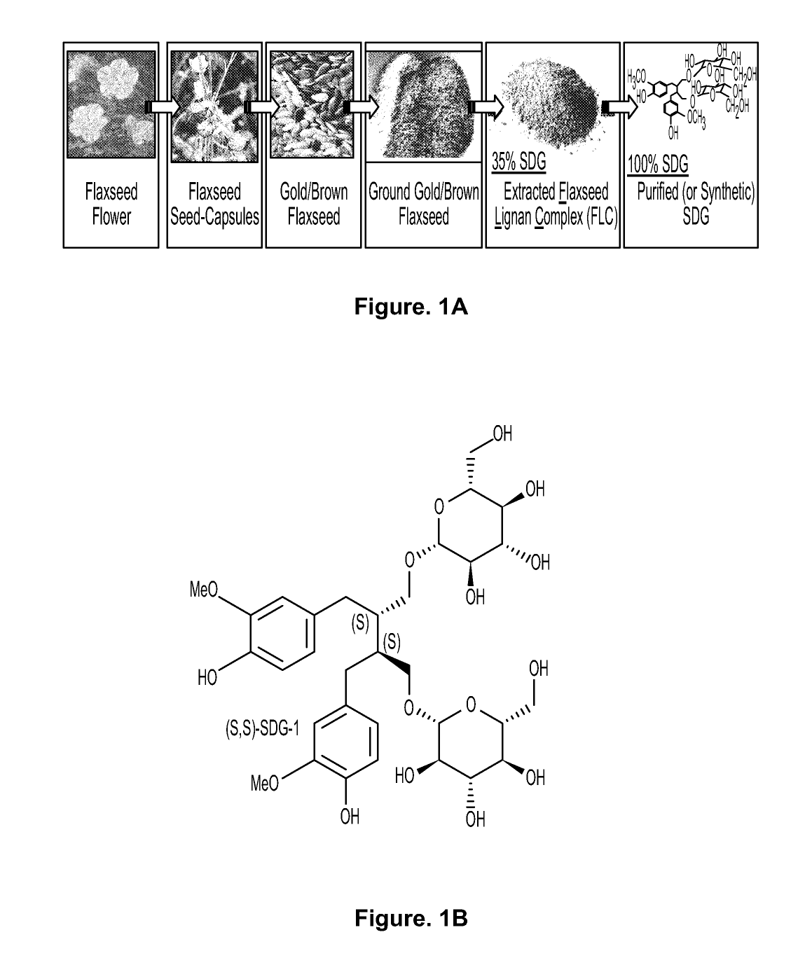

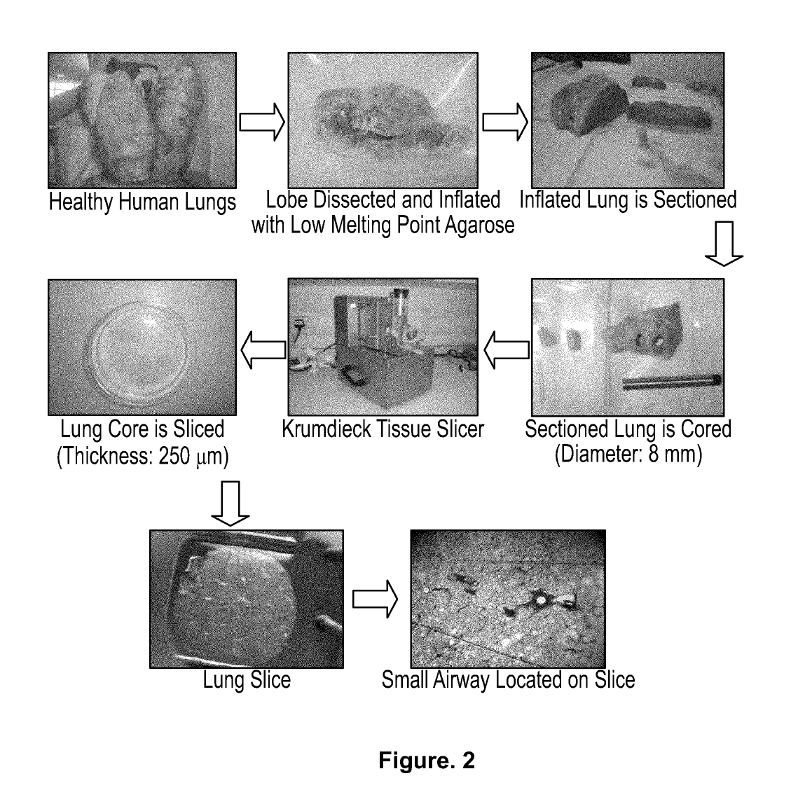

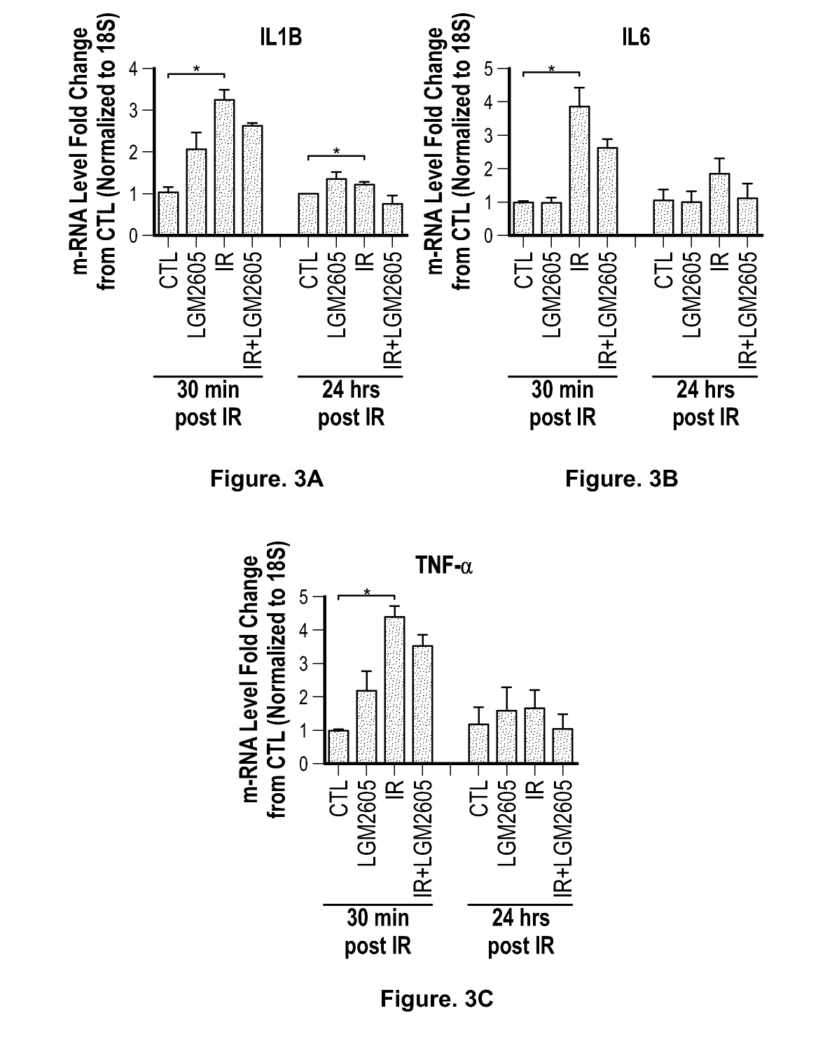

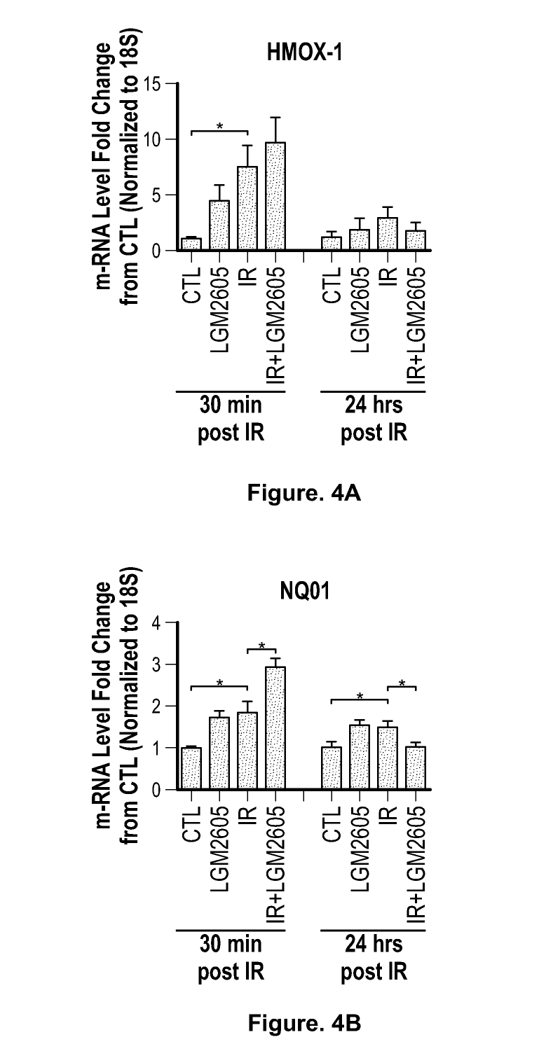

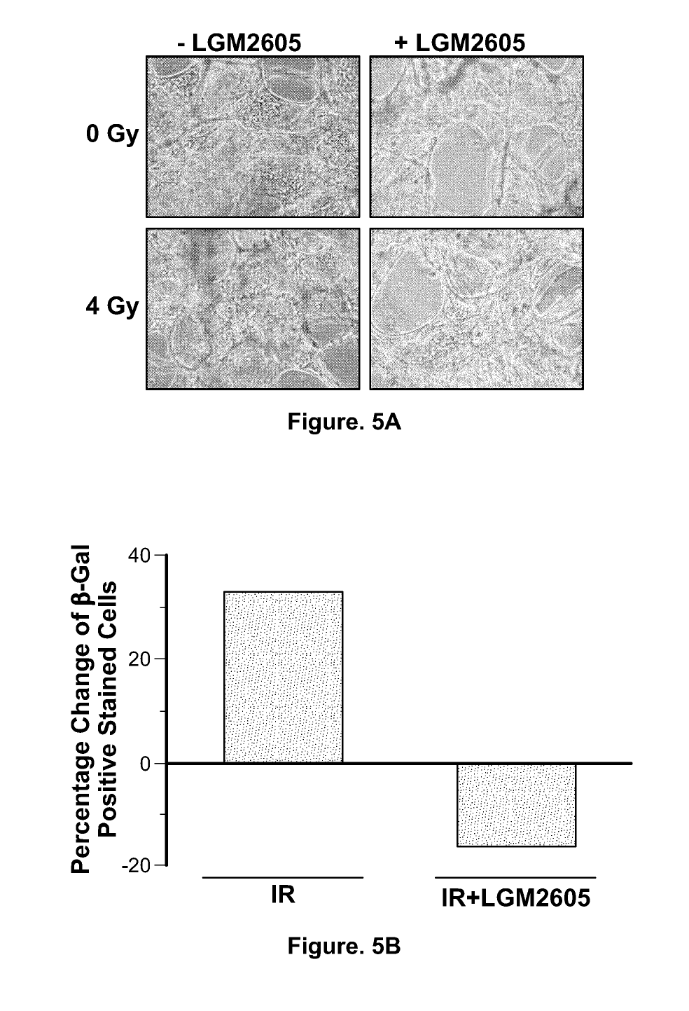

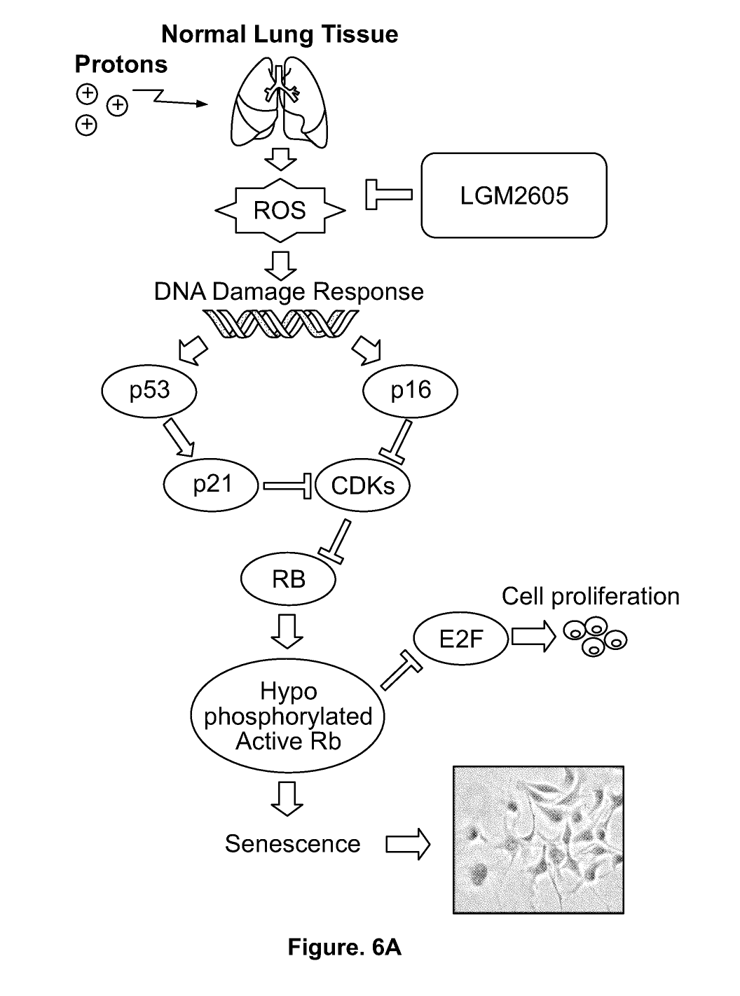

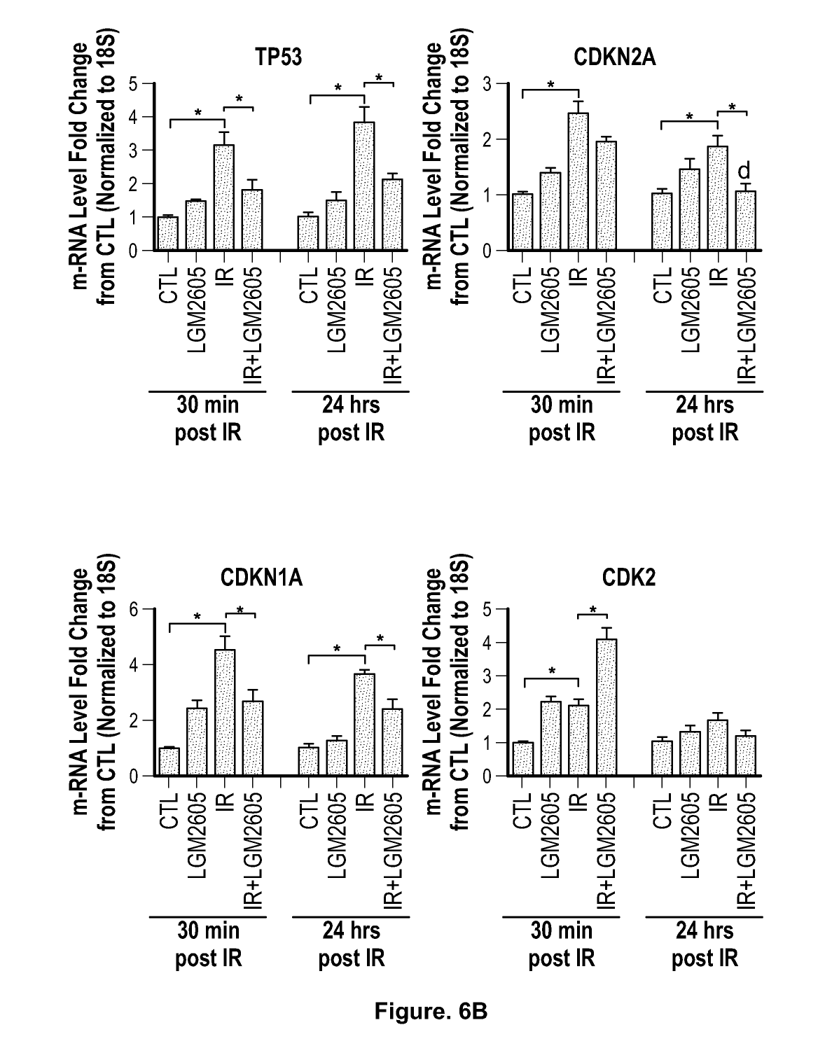

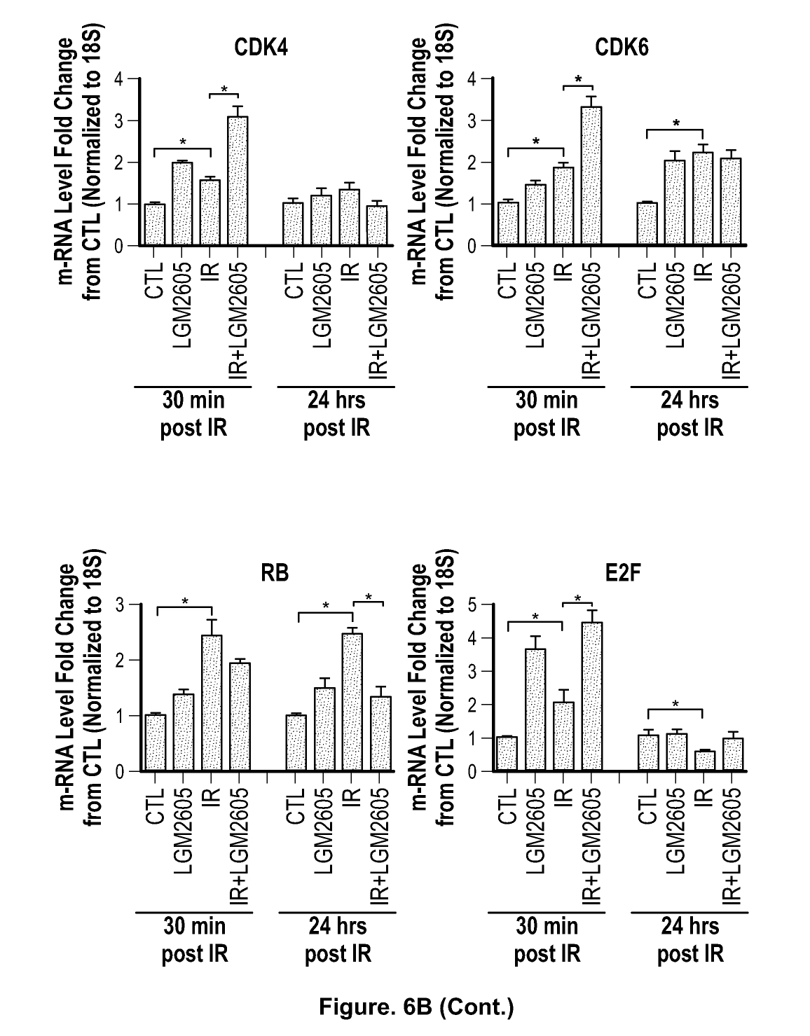

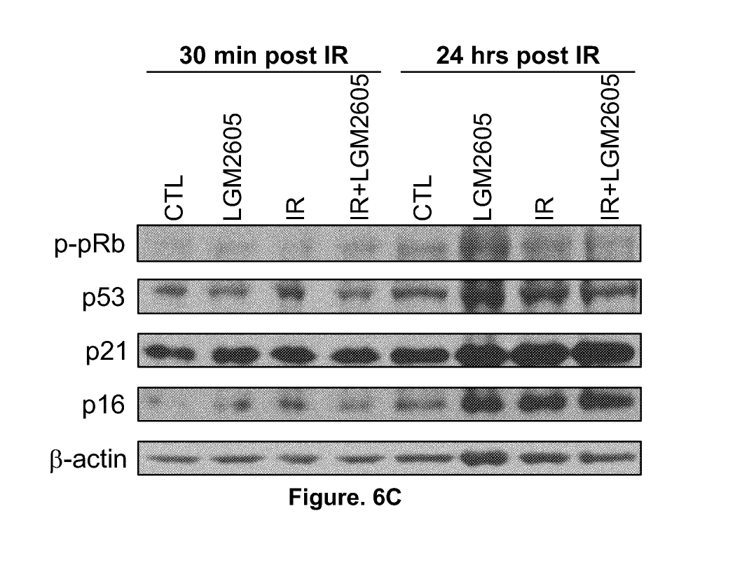

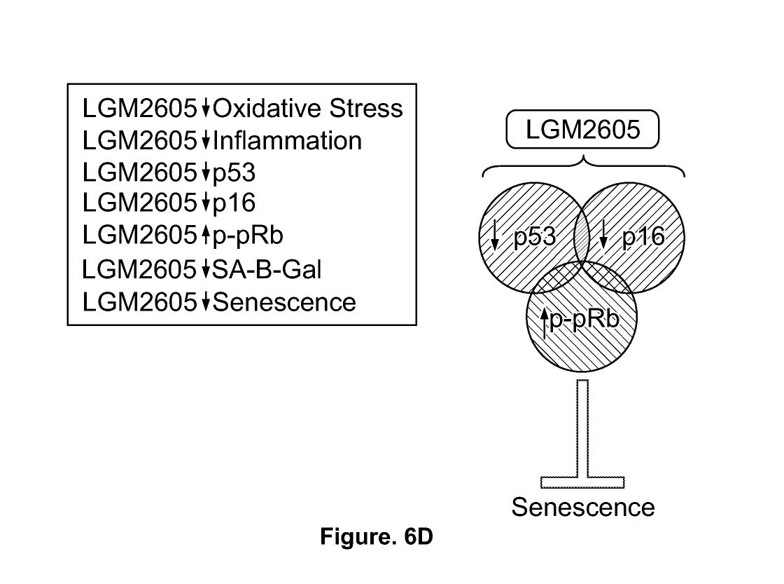

### DETAILED DESCRIPTION OF THE INVENTION

The invention relates to the use of secoisolariciresinol diglucoside (SDG), obtained from natural sources, such as flaxseed, or generated synthetically (synthetic SDG is also referred to herein as LGM 2605), other active components in flaxseed, and related compounds for treating proton radiation associated lung injury and protecting normal lung tissue against proton radiation exposure. The invention also relates to the use of SDG, other active components in flaxseed, and related compounds in down-regulating senescence markers, and thereby protecting from senescence associated aging phenotypes.

Surprisingly and unexpectedly, the inventors of this application have found that SDG can be used to treat proton radiation associated lung injury and protect normal lung tissue against proton radiation exposure. The inventors have also found that SDG can be used in down-regulating senescence markers, and thereby protecting from senescence associated or radiation induced aging phenotypes.

In one aspect, provided herein are therapeutic and prophylactic methods of using SDG, or its isomers for radioprotection and other uses such as, for example, treating senescence associated or radiation induced aging phenotypes.

SDG can be isolated from natural sources or chemically synthesized. Due to complex extraction, purification and enrichment methods to isolate secoisolariciresinol diglucoside (SDG) from natural resources, in a preferred embodiment, SDG is chemically synthesized.

Using the natural compounds vanillin and glucose, two enantiomers (their structures are depicted below) of SDG: SDG (S,S) and SDG (R,R), were successfully synthesized (Mishra et al., Bioorganic & Medicinal Chemistry Letters 2013, (19):5325).

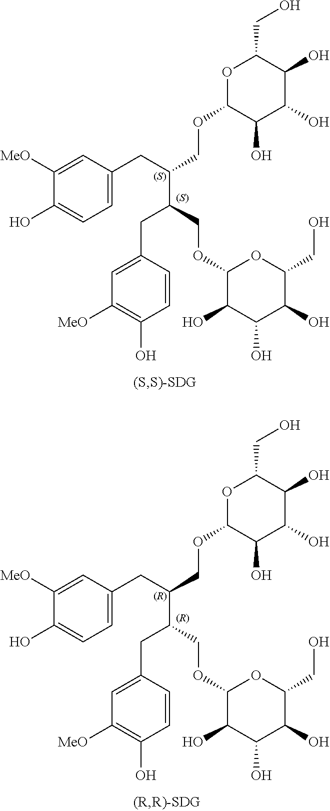

In one embodiment, SDG is SDG (S,S). In another embodiment, SDG is SDG (R,R).

In another aspect, other bioactive ingredients of flaxseed, their metabolites, their degradants or stereoisomers can also be used. Examples of the other bioactive ingredients of flaxseed include, for example, but not limited to, secoisolariciresinol (SECO), enterodiol (ED), enterolactone (EL), analogs thereof, isomers (including stereoisomers) thereof, or a combination thereof.

Flaxseed, its bioactive ingredients, and its metabolites are known in the art and described in U.S. Patent Publication Nos. 2010/0239696; 2011/0300247; and 2014/0308379; and in International Patent Publication No. WO2014/200964, each of which is incorporated by reference herein in its entirety. In another aspect, flaxseed extract can be used.

The primary lignan found in flaxseed is 2,3-bis (3-methoxy-4-hydroxybenzyl) butane-1,4-diol (secoisolariciresinol or SECO), which is stored as the conjugate SDG in its native state in the plant. SDG is metabolized in the human intestine to enterodiol (ED), and enterolactone (EL). Synthetic analogs of enterodiol and enterolactone are known (see, e.g., Eklund et al., Org. Lett., 2003, 5:491).

A “metabolite” is a substance produced by metabolism or by a metabolic process. For example, a metabolite of SDG is EL or ED. A “degradant” is a product of the breakdown of a molecule, such as SDG, into smaller molecules. It will be appreciated by one skilled in the art that a metabolite or a degradant may be a chemically synthesized equivalent of a natural metabolite or degradant.

An “analog” is a compound whose structure is related to that of another compound. The analog may be a synthetic analog.

An “ingredient” or “component” is an element or a constituent in a mixture or compound.

In another aspect, the invention relates to a pharmaceutical composition. “Pharmaceutical composition” refers to an effective amount of an active ingredient, e.g., (S,S)-SDG (R,R)-SDG, meso-SDG, SDG, SECO, EL, ED and analogs thereof, together with a pharmaceutically acceptable carrier or diluent.

The compositions described herein may include a “therapeutically effective amount.” A “therapeutically effective amount” refers to an amount effective, at dosages and for periods of time necessary, to achieve the desired therapeutic result. A therapeutically effective amount may vary according to factors such as the disease state, age, sex, and weight of the individual, and the ability of the composition to elicit a desired response in the individual. A therapeutically effective amount is also one in which toxic or detrimental effects of the molecule are outweighed by the therapeutically beneficial effects.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

The pharmaceutical compositions can be administered to a subject by any suitable method known to a person skilled in the art, such as orally, parenterally, transmucosally, transdermally, intramuscularly, intravenously, intra-dermally, subcutaneously, intra-peritonealy, intra-ventricularly, intra-cranially, intra-vaginally, intra-tumorally, or bucally. Controlled release may also be used by embedding the active ingredient in an appropriate polymer which may then be inserted subcutaneously, intratumorally, bucally, as a patch on the skin, or vaginally. Coating a medical device with the active ingredient is also covered.

In some embodiments, the pharmaceutical compositions are administered orally, and are thus formulated in a form suitable for oral administration, i.e., as a solid or a liquid preparation. Suitable solid oral formulations include tablets, capsules, pills, granules, pellets and the like. Suitable liquid oral formulations include solutions, suspensions, dispersions, emulsions, oils and the like. In some embodiments, the active ingredient is formulated in a capsule. In accordance with this embodiment, the compositions of the present invention comprise, in addition to the active compound and the inert carrier or diluent, drying agent, in addition to other excipients as well as a gelatin capsule.

In some embodiments, the pharmaceutical compositions are administered by intravenous, intra-arterial, or intra-muscular injection of a liquid preparation. In some embodiments, the pharmaceutical composition is a liquid preparation formulated for oral administration. In some embodiments, the pharmaceutical composition is a liquid preparation formulated for intravaginal administration. Suitable liquid formulations include solutions, suspensions, dispersions, emulsions, oils and the like. In some embodiments, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In some embodiments, the pharmaceutical compositions are administered intra-muscularly and are thus formulated in a form suitable for intra-muscular administration. In some embodiments, the pharmaceutical compositions are administered intra-bucally and are thus formulated in a form suitable for buccal administration.

In some embodiments, the pharmaceutical compositions are administered topically to body surfaces and are thus formulated in a form suitable for topical administration. Suitable topical formulations include gels, ointments, creams, lotions, drops, controlled release polymers and the like. For topical administration, the flaxseed, its bioactive ingredient, or a metabolite thereof is prepared and applied as a solution, suspension, or emulsion in a physiologically acceptable diluent with or without a pharmaceutical carrier.

In some embodiments, the pharmaceutical compositions provided herein are controlled-release compositions, i.e. compositions in which the flaxseed, its bioactive ingredient, or a metabolite thereof is released over a period of time after administration. Controlled- or sustained-release compositions include formulation in lipophilic depots (e.g. fatty acids, waxes, oils). In other embodiments, the composition is an immediate-release composition, i.e. a composition in which all the flaxseed, its bioactive ingredient, or a metabolite thereof is released immediately after administration.

In some embodiments, compositions for use in the methods provided herein are administered at a therapeutic dose once per day. In some embodiments, the compositions are administered once every two days, twice a week, once a week, or once every two weeks.

Techniques for extracting and purifying SDG are known in the art and described in U.S. Pat. No. 5,705,618, which is incorporated herein by reference. Techniques for synthesizing SDG, its stereoisomers and analogs are described in Mishra O P, et al., Bioorganic & Medicinal Chemistry Letters 2013, (19):5325-5328 and in International Patent Publication No. WO2014/200964, which are hereby incorporated by reference in their entireties. Bioactive components for use in the methods provided herein may also be chemically synthesized directly into the mammalian, readily metabolizable forms, Enterodiol (ED) or Enterolactone (EL), as is known in the art.

(S,S)-SDG (R,R)-SDG, (S,R)-SDG (R,S)-SDG, meso-SDG, SECO, EL, ED or an analog thereof may be administered at a dose of 0.1 ng/kg to 500 mg/kg.

The treatment with (S,S)-SDG (R,R)-SDG, (S,R)-SDG (R,S)-SDG, meso-SDG, SDG, SECO, EL, ED or an analog thereof is a single administration to several days, months, years, or indefinitely.

As used herein, “treating” may refer to either therapeutic treatment or prophylactic or preventative measures, wherein the object is to prevent or lessen the targeted pathologic condition or disorder as described herein, or both. Therefore, compositions for use in the methods provided herein may be administered to a subject before the exposure, e.g., to radiation, a carcinogen, a toxicant, or hypochlorite ions. In some cases, compositions for use in the methods provided herein may be administered to a subject after the exposure. Thus treating a condition as described herein may refer to preventing, inhibiting, or suppressing the condition in a subject.

Furthermore, as used herein, the terms “treat” and “treatment” refer to therapeutic treatment, as well prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already having been exposed, e.g., to radiation, a carcinogen, a toxicant, or hypochlorite ions, as well as those prone to being exposed or those expecting to be exposed.

The diseases or conditions that can be treated or prevented by the compositions of the invention include, for example, but are not limited to radiation damage (e.g., proton radiation damage in lungs), a senescent phenotype (e.g., proton radiation associated senescent phenotype), a senescence associated aging disease or condition, radiation associated aging disease or condition (e.g., proton radiation associated aging disease or condition), and signs of aging (e.g., aging of skin).

In one example, subjects in need of radioprotection or radiation mitigation according to methods provided herein are subjects who will, are, or have been exposed to potentially deleterious amounts of radiation. It will be understood that such exposure may be a single exposure, periodic exposure, sporadic exposure or ongoing exposure to the radiation. It is also understood that such radiation exposure includes accidental exposure, incidental or intentional exposure.

Examples of subjects who may be in need of radioprotection or radiation mitigation according to the methods of the present invention include but are not limited to, patients who are exposed to radiation (e.g., proton radiation, photon radiation) as part of therapeutic regimen (e.g., cancer patients who require radiation therapy), subjects who are exposed to radiation for to diagnose a disease or condition (e.g., subjects receiving dental or bone X-rays, patients receiving PET scans, CT scans and the like). Examples of subjects who may be in need of radioprotection or radiation mitigation according to the methods of the present invention also include those who may be exposed to radiation as a result of their profession or life style choices (e.g., airplane flight crews or other frequent air travelers, and even space travelers, who are exposed to higher than average radiation levels; laboratory technicians and other workers; or those exposed through the use of electronic devices) or those exposed to accumulations of radon (e.g., accumulations in dwellings or mines) or outdoor workers or sunbathers exposed to natural radiation from the sun. Other subjects who may be in need of radioprotection according to the methods of the present invention include those who are accidentally exposed to radiation, such as leaks or spills, (e.g., nuclear reactor leaks or accidents or laboratory spills). Also contemplated are those exposed to radiation from the detonation of a nuclear warhead, as a result of war or terrorism. Additional subjects encompassed are those who are exposed to a terrorist's detonation of conventional explosives that disperse radioactive materials.

In some embodiments, subjects in need of treatment and the methods and compositions described herein may include, but are not limited to, subjects with aging disease or condition, subjects with senescence associated phenotypes, subjects with radiation induced aging phenotypes, and subjects with cosmetic skin conditions (e.g., wrinkles and age spots).

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In addition to humans, the subject may include dogs, cats, pigs, cows, sheep, goats, horses, buffalo, ostriches, guinea pigs, rats, mice, birds (e.g., parakeets) and other wild, domesticated or commercially useful animals (e.g., chicken, geese, turkeys, fish). The term “subject” does not exclude an individual that is normal in all respects. The term “subject” includes, but is not limited to, a human in need of therapy for, or susceptible to, a condition or its sequelae.

Any patent, patent application publication, or scientific publication, cited herein, is incorporated by reference herein in its entirety.

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Thus these examples should in no way be construed, as limiting the broad scope of the invention.

### EXAMPLES

### Synthetic Secoisolariciresinol Diglucoside (LGM2605) Protects Human Precision-Cut Lung Slices (huPCLS) From Proton Radiation Damage

Lung cancer remains the leading cause of death from cancer in the US and worldwide. Radiation therapy plays a prominent role in the treatment of patients with non-metastatic disease. The introduction of proton beam, in the field of radiation therapy, led to the reduction of the dose received by the tumor-surrounding normal tissue while allowed for maintained and more focused doses to the tumor target, leading to their successful shrinkage. Nevertheless, a substantial risk of late side effects in long-term survivors, such as secondary cancers and significant normal tissue damage, still remains.

Using a mouse model of proton radiation exposure, we have identified dose-dependent pathophysiological changes in the lung. We also established the lignan component of flaxseed consisting mainly of secoisorariciresinol diglucoside (SDG), as a potent protector from radiation-induced lung toxicity. We have chemically synthesized SDG (LGM2605) and have shown equipotent activity with its natural counterpart. Thus, we further hypothesize that LGM2605 will be effective against proton-induced pulmonary damage, such as inflammation, oxidative stress and senescence.

Methods: Since the testing of our hypothesis in human tissues is critical and cannot be mechanistically pursued in patients, we pursued a novel approach, using an ex vivo model of human normal lung precision-cut sections (huPCLS), used for the first time to our knowledge, in radiation studies. The huPCLS consist of all relevant cell types of the respiratory tract, situated in their microanatomical environment, validated for pharmacological testing. Human lung sections were exposed to 4 Gy proton radiation in the presence of 0 and 50 Mm LGM2605, given 4 hours prior to exposure. Tissues were evaluated 30 min and 24 hours post irradiation for gene expression, protein level changes, and betagalactosidase activity, relevant to inflammation, oxidative stress and senescence.

Results: We identified an LGM2605-mediated reduction of inflammatory markers such as IL-1β and IL-6 and TNFα, cell cycle-related p53 and p16 and an increase of the antioxidant HO and NQO1, after proton radiation. B-gal staining further complements the anti-senescent profile of SDG activity, after radiation exposure.

LGM2605 upregulates antioxidant genes and downregulates proinflammatory cytokine gene levels, after 4 Gy proton radiation of the huPCLS, such as IL6, IL1B and TNF-α.

LGM2605 reduces gene and protein level of senescent markers of huPCLS exposed to proton radiation, such as TP53, CDKN2A, p53, p16.

LGM2605 protects huPCLS from a senescent-like phenotype, induced by proton radiation, such as the high level of SA-β-galactosidase positive staining.

These results demonstrate that SDG possesses promising properties as a protector of normal tissue against proton radiation exposure. Additionally, these results demonstrate that SDG is an anti-aging agent and can be used to treat or prevent a senescent phenotype.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for treating or preventing a senescent phenotype in a subject in need thereof, the method comprising: administering to said subject an effective amount of secoisolaricirecinol diglucoside (SDG), an analog thereof, a stereoisomer thereof, or a combination thereof, thereby treating said senescent phenotype in said subject.

2. The method of claim 1, wherein said SDG is (S,S)-SDG.

3. The method of claim 1, wherein said SDG is (R,R)-SDG.

4. The method of claim 1, wherein said SDG is a synthetic SDG.

5. The method of claim 1, wherein said SDG is an SDG analog.

6. The method of claim 1, wherein said SDG is administered in a dietary composition.

7. The method of claim 1, wherein said step of administering is performed orally.

8. The method of claim 1, wherein said SDG is administered at a concentration from about 1 nanomolar (nM) to about 1 molar (M).

9. The method of claim 8, wherein the said SDG is administered at a concentration from about 25 μM to about 250 μM.

10. The method of claim 1, wherein the subject is a human subject.

11. The method of claim 1, wherein said senescent phenotype is associated with an aging disease or condition.

12. The method of claim 1, wherein said senescent phenotype is induced by radiation.

13. The method of claim 1, wherein said senescent phenotype is induced by proton radiation.

14. The method of claim 1, wherein the administration of said SDG upregulates the level of an antioxidant gene and down regulates the level of a proinflammatory cytokine gene.

15. The method of claim 14, wherein the antioxidant gene is HMOX-1, NQO1, or a combination thereof.

16. The method of claim 14, wherein the proinflammatory cytokine gene is IL6, IL1B, TNF-alpha or a combination thereof.

17. The method of claim 1, wherein the administration of said SDG reduces nucleic acid and protein levels of a senescent marker.

18. The method of claim 17, wherein the senescent marker is TP53, CDKN2A, p53, p16, or a combination thereof.

19. A method for treating or preventing a senescence associated aging disease or condition in a subject in need thereof, the method comprising: administering to said subject an effective amount of secoisolaricirecinol diglucoside (SDG), an analog thereof, a stereoisomer thereof, or a combination thereof, thereby treating said senescence associated aging disease or condition in said subject.

20. A method for treating or preventing radiation damage in a subject who has been or will be exposed to proton radiation, the method comprising: administering to said subject an effective amount of secoisolaricirecinol diglucoside (SDG), an analog thereof, a stereoisomer thereof, or a combination thereof.

21. The method of claim 20, wherein said SDG is (S,S)-SDG.

22. The method of claim 20, wherein said SDG is (R,R)-SDG.

23. The method of claim 20, wherein said SDG is a synthetic SDG.

24. The method of claim 20, wherein said SDG is an SDG analog.

25. The method of claim 20, wherein said SDG is administered in a dietary composition.

26. The method of claim 20, wherein said step of administering is performed orally.

27. The method of claim 20, wherein said SDG is administered at a concentration of about 1 nanomolar (nM) to about 1 molar (M).

28. The method of claim 27, wherein the said SDG is administered at a concentration of about 25 μM to about 250 μM.

29. The method of claim 20, wherein the subject is a human subject.

30. The method of claim 20, wherein said radiation is associated with a medical treatment.

31. The method of claim 20, wherein said radiation is associated with a radiation cancer therapy.

32. The method of claims 20, wherein the subject has been or will be exposed to radiation as part of a therapeutic procedure.

33. The method of claim 20, wherein the subject is a cancer patient who has or will receive radiotherapy.

34. The method of claim 33, wherein the cancer patient is a lung cancer patient.

35. The method of claim 20, wherein the subject has been or will be exposed to radiation as part of a diagnostic procedure.

36. The method of claim 35, wherein the diagnostic procedure is a dental or bone X-ray.

37. The method of claim 35, wherein the diagnostic procedure comprises a PET or CT scan.

38. The method of claim 20, wherein the subject has been accidentally exposed to radiation.

39. The method of claim 20, wherein the subject has been or will be exposed to radiation as part of their occupation.

40. The method of claim 39, wherein the subject's occupation is as a laboratory technician.

41. The method of claim 20, wherein the subject has been exposed to radon.

42. The method of claim 20, wherein the subject has been exposed to radiation as a result of terrorism.

43. The method of claim 20, wherein the administration of said SDG upregulates the level of an antioxidant gene and down regulates the level of a proinflammatory cytokine gene.

44. The method of claim 43, wherein the antioxidant gene is HMOX-1, NQO1, or a combination thereof.

45. The method of claim 43, wherein the proinflammatory cytokine gene is IL6, IL1B, TNF-alpha or a combination thereof.

46. The method of claim 20, wherein the administration of said SDG reduces nucleic acid and protein levels of a senescent marker.

47. The method of claim 46, wherein the senescent marker is TP53, CDKN2A, p53, p16, or a combination thereof.

48. A method for treating or preventing proton radiation associated lung injury in a subject in need thereof, the method comprising: administering to said subject an effective amount of secoisolaricirecinol diglucoside (SDG), an analog thereof, a stereoisomer thereof, or a combination thereof, thereby treating or preventing said proton radiation associated lung injury in said subject.

49. A method for protecting normal lung tissue against proton radiation exposure in a subject in need thereof, the method comprising: administering to said subject an effective amount of secoisolaricirecinol diglucoside (SDG), an analog thereof, a stereoisomer thereof, or a combination thereof, thereby protecting normal lung tissue against proton radiation exposure in said subject.

50. A method for protecting a biomolecule, a cell, or a tissue from damage by proton radiation in a subject in need thereof, the method comprising: administering to said subject an effective amount of secoisolaricirecinol diglucoside (SDG), an analog thereof, a stereoisomer thereof, or a combination thereof.

51. The method of any one of claims 1-25, wherein the biomolecule is a nucleic acid.

52. The method of any one of claims 1-25, wherein the biomolecule is a protein or a lipid.

53. A method for treating or preventing a senescent phenotype in a subject in need thereof, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

54. A method for treating or preventing a senescence associated aging disease or condition in a subject in need thereof, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

55. A method for treating or preventing radiation damage in a subject who has been or will be exposed to radiation, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

56. A method for treating or preventing proton radiation associated lung injury in a subject in need thereof, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

57. A method for protecting normal lung tissue against proton radiation exposure in a subject in need thereof, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

58. A method for protecting a biomolecule, a cell, or a tissue from damage by proton radiation in a subject in need thereof, the method comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

59. A method for treating or preventing a senescent phenotype in a subject in need thereof, the method comprising: administering to said subject an effective amount of a flax seed extract.

60. A method for treating or preventing a senescence associated aging disease or condition in a subject in need thereof, the method comprising: administering to said subject an effective amount of a flax seed extract.

61. A method for treating or preventing radiation damage in a subject who has been or will be exposed to radiation, the method comprising: administering to said subject an effective amount of a flax seed extract.

62. A method for treating or preventing proton radiation associated lung injury in a subject in need thereof, the method comprising: administering to said subject an effective amount of a flax seed extract.

63. A method for protecting normal lung tissue against proton radiation exposure in a subject in need thereof, the method comprising: administering to said subject an effective amount of a flax seed extract.

64. A method for protecting a biomolecule, a cell, or a tissue from damage by proton radiation in a subject in need thereof, the method comprising: administering to said subject an effective amount of a flax seed extract.
